(19) United States
(12) Patent Application Publication
(10) Pub. No.: US 2022/0090091 A1
(43) Pub. Date: Mar. 24, 2022

(54) COMPOSITIONS OF POLYNUCLEIC ACID VECTORS AND USES THEREOF

(71) Applicant: Asimov, Inc., Boston, MA (US)

(72) Inventors: Alec A.K. Nielsen, Boston, MA (US); Yekaterina Tarasova, Boston, MA (US); Amber Marie Delhagen, Boston, MA (US)

(73) Assignee: Asimov, Inc., Boston, MA (US)

(21) Appl. No.: 17/421,596

(22) PCT Filed: Jan. 7, 2020
(86) PCT No.: PCT/US2020/012494

§ 371 Date: Jul. 8, 2021

Related U.S. Application Data
(60) Provisional application No. 62/790,343, filed on Jan. 9, 2019.

(51) Int. Cl. C12N 15/66; C12N 15/10; C12N 15/11

(52) U.S. Cl. CPC C12N 15/66; C12N 15/10; C12N 2310/3521; C12N 2800/70; C12N 15/11

(57) ABSTRACT

Disclosed herein are compositions of destination and entry vectors. Also disclosed herein are the use of destination and entry vectors in methylation-obstructed assembly reactions, wherein the assembled sequences may be used as entry vectors in subsequent assembly reactions.

### RELATED APPLICATION

This application claims the benefit under 35 U.S.C. § 119(e) of U.S. provisional application Ser. No. 62/790,343, filed Jan. 9, 2019, the entire contents of which are incorporated by reference herein.

### FIELD

Disclosed herein are compositions of destination and entry vectors. Also disclosed herein are the use of destination and entry vectors in methylation-obstructed assembly reactions, wherein the assembled sequences may be used as entry vectors in subsequent assembly reactions.

### BACKGROUND

Various cloning strategies have previously been described. Traditionally, restriction ligation cloning was used to insert a nucleic acid fragment of interest into a vector. Because of the limitations of this traditional approach—such as those relating to the restriction enzyme cleavage site, the introduction of “scars,” the choice of restriction enzymes, and inefficiencies in cloning—other approaches were developed to clone larger polynucleic acids in a hierarchical fashion. For example, Type IIS hierarchical cloning strategies include MoClo (Addgene) and Golden Gate (NEB) and recombination-based hierarchical cloning strategies include Gateway Cloning (Thermo).

### SUMMARY

In some aspects, the disclosure relates to destination vectors. In some embodiments, a polynucleic acid destination vector comprising a backbone component and an insertion site component, wherein: (a) the backbone component comprises a nucleic acid sequence of a selectable marker, an origin of replication, and at least one Type IIS restriction site comprising a common recognition site and corresponding cleavage site, wherein the common recognition site is overlapped by a methylation site; and (b) the insertion site component comprises a 5′ Type IIS dual restriction site and a 3′ Type IIS dual restriction site, optionally, wherein the 5′ and 3′ Type IIS dual restriction sites are separated by at least one nucleotide; wherein each Type IIS dual restriction site comprises: (i) a first common recognition site and corresponding cleavage site, wherein the first common recognition site is overlapped by a methylation site that forms the border between the insertion site component and the backbone component and (ii) a second common recognition site and corresponding cleavage site, wherein the second recognition site lacks an overlapping methylation site, wherein the cleavage site corresponding to the first common recognition site and the cleavage site corresponding to the second common recognition site are both positioned between the first common recognition site and the second common recognition site, wherein: methylation of the destination vector at common recognition sites overlapped by a methylation site, blocks cleavage of the cleavage site corresponding to that common recognition site; exposure of the destination vector, when methylated, to a Type IIS restriction enzyme that recognizes the common recognition sites of the destination vector, generates two polynucleic acid fragments, wherein the terminal 5′ or 3′ nucleic acid overhangs of the fragment comprising the backbone component differ in nucleotide sequence; and the Type IIS cleavage sites in (a) differ from the Type IIS cleavage sites in (b) in nucleotide sequence.

In some embodiments, the cleavage site corresponding to the first common recognition site and the cleavage site corresponding to the second common recognition site of the 5′ Type IIS dual restriction site or the 3′ Type IIS dual restriction site are separated from each other by at least one nucleotide. In some embodiments, the cleavage site corresponding to the first common recognition site and the cleavage site corresponding to the second common recognition site of both the 5′ Type IIS dual restriction site and the 3′ Type IIS dual restriction site are separated from each other by at least one nucleotide.

In some embodiments, the cleavage site corresponding to the first common recognition site and the cleavage site corresponding to the second common recognition site of the 5′ Type IIS dual restriction site and the cleavage site corresponding to the first common recognition site and the cleavage site corresponding to the second common recognition site of the 3′ Type IIS dual restriction site are separated from each other by differing nucleotide sequences. In some embodiments, the differing nucleotide sequences comprise differing nucleic acid lengths.

In some embodiments, the cleavage site corresponding to the first common recognition site and the cleavage site corresponding to the second common recognition site of the 5′ Type IIS dual restriction site and the cleavage site corresponding to the first common recognition site and the cleavage site corresponding to the second common recognition site of the 3′ Type IIS dual restriction site are separated from each other by an identical nucleotide sequence.

In some embodiments, the cleavage site corresponding to the first common recognition site and the cleavage site corresponding to the second common recognition site of the 5′ Type IIS dual restriction site and/or the 3′ Type IIS dual restriction site is a shared cleavage site.

In some embodiments, the 5′ Type IIS dual restriction site and a 3′ Type IIS dual restriction site are separated by a nucleic acid sequence encoding a visual readout or suicide cassette. In some embodiments, the visual readout is selected from the group consisting of a fluorescent protein, a chromogenic protein, LacZ, or LacZa. In some embodiments, the suicide cassette comprises the nucleic acid sequence of ccdB.

In some embodiments, the Type IIS restriction enzyme that binds to the common recognition site is selected from the group consisting of BsaI, BsmBI, BtgZI, Esp3I, FokI, HphI, BcgI, AlwI, MboII, MmeI, BsmFI, BceAI, BcoDI, BfuAI, BsmAI, EarI, EciI, FauI, HgaI, HpyAV, PleI, BbsI, SapI, and SfaNI. In some embodiments, the Type IIS restriction enzyme is a high fidelity restriction enzyme. In some embodiments, at least one Type IIS restriction site in the backbone component of (a) is located within or flanking the selectable marker or the origin of replication. In some embodiments, the cleavage site of at least one Type IIS restriction site in the backbone component of (a) comprises a low ligation efficiency sequence content. In some embodiments, the selectable marker comprises an antibiotic resistance gene.

In some embodiments, the methylation sites of (a) and (b) are methylated by the same methyltransferase.

In some embodiments, the methyltransferase is selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

In some embodiments, exposure of the destination vector, when unmethylated, to a Type IIS restriction enzyme that recognizes the common recognition sites of the destination vector generates at least three polynucleic acid fragments, wherein each polynucleic acid comprises terminal 5′ or 3′ nucleic acid overhangs, and wherein the terminal 5′ or 3′ nucleic acid overhangs of the fragment comprising the second common recognition sequence of both the 5′ Type IIS dual restriction site and the 3′ Type IIS dual restriction site differ in nucleotide sequence.

In other aspects, the disclosure relates to entry vectors. In some embodiments, a polynucleic acid entry vector comprises a backbone component and an insert component, wherein (a) the backbone component comprises the backbone component of a polynucleic acid destination vector as disclosed herein; and (b) the insert component comprises from 5′ to 3′, a first Type IIS restriction site, an insert, and a second Type IIS restriction site; wherein the first and second Type IIS restriction sites each comprises: (i) a common recognition site overlapped by a methylation site, wherein the methylation site forms the border between the insert component and the backbone component and (ii) a corresponding cleavage site, wherein cleavage of the cleavage site of the first and second Type IIS restriction sites generates 5′ or 3′ overhangs, wherein the 5′ or 3′ overhang of the first Type IIS restriction site and the 5′ or 3′ overhang of the second Type IIS restriction sites differ in nucleotide sequence from each other. In some embodiments, the insert is a nucleic acid sequence that is to be combined in an assembly reaction.

In yet other aspects, the disclosure relates to methods of assembling polynucleic acids into a predefined sequence. In some embodiments, the method comprises: (a) forming a reaction mixture by combining: (i) a destination vector as disclosed herein, wherein the methylation sites of both the backbone component and the insertion site component of the destination vector are methylated; (ii) at least one entry vector as disclosed herein, wherein the methylation sites of the backbone component and the insert component of each of the at least one entry vectors are unmethylated; (iii) a Type IIS restriction enzyme, wherein the Type IIS restriction enzyme recognizes the common recognition sites of the destination vector and entry vector; and (iv) a ligase; (b) incubating the reaction mixture for a time sufficient for Type IIS restriction enzyme-mediated cleavage of the destination vector and the at least one entry vectors; and (c) incubating the reaction mixture for a time sufficient for the ligase to ligate the insert of each of the at least one entry vector into the backbone component of the destination vector, thereby generating a circular polynucleic acid; and wherein the 5′ or 3′ overhangs of the backbone component of the destination vector and the insert component of each of the at least one entry vector uniquely complement one another so as to form a predefined sequence comprising the backbone component of the destination vector of step (a)(i) and the insert component of each of the at least one entry vectors of step (a)(ii).

In some embodiments, the destination vector is methylated in vitro. In some embodiments, the destination vector is methylated in vivo. In some embodiments, the destination vector is methylated in a bacterial strain that expresses a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

In some embodiments, the method further comprises isolating the ligated destination vector containing the insert from the other components of the reaction mixture. In some embodiments, the ligated destination vector is isolated by transforming bacteria with the reaction mixture and screening the bacteria for the presence of the correctly ligated assembly.

In some embodiments, the method further comprises demethylating the isolated ligated destination vector to generate a second entry vector. In some embodiments, the isolated ligated destination vector is demethylated passively in vivo through replication in a bacterial strain that lacks a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

In other aspects, the disclosure relates to methods of cloning a nucleic acid sequence of interest. In some embodiments, the method comprises: (a) forming a reaction mixture by combining: (i) a destination vector as disclosed herein, wherein the methylation sites of the backbone component are methylated; (ii) at least one polynucleic acid fragment, wherein each polynucleic acid fragment comprises an internal sequence flanked by a common recognition site and corresponding cleavage site at both ends; (iii) a Type IIS restriction enzyme, wherein the Type IIS restriction enzyme recognizes the common recognition sites of the destination vector and the at least one polynucleic acid fragment; and (iv) a ligase; (b) incubating the reaction mixture for a time sufficient for Type IIS restriction enzyme-mediated cleavage of the destination vector and the at least one polynucleic acid fragment; and (c) incubating the reaction mixture for a time sufficient for the ligase to ligate the internal nucleic acid sequence of each of the at least one polynucleic acid fragments into the backbone component of the destination vector, thereby generating a circular polynucleic acid; and wherein the internal sequences of the at least one polynucleic acid fragment comprises a nucleic acid sequence of interest; and wherein the 5′ or 3′ overhangs of the backbone component of the destination vector and each internal sequence of the at least one polynucleic acid fragment uniquely complement one another so as to form a predefined sequence comprising the backbone component of the destination vector of step (a)(i) and the nucleic acid sequence of interest.

In some embodiments, the destination vector is methylated in vitro. In some embodiments, the destination vector is methylated in vivo. In some embodiments, the destination vector is methylated in a bacterial strain that expresses a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

In some embodiments, the method further comprises isolating the ligated destination vector containing the insert from the other components of the reaction mixture. In some embodiments, the ligated destination vector is isolated by transforming bacteria with the reaction mixture and screening the bacteria for the presence of the correctly ligated assembly.

In some embodiments, the method further comprises demethylating the isolated ligated destination vector to generate a second entry vector. In some embodiments, the isolated ligated destination vector is demethylated passively in vivo through replication in a bacterial strain that lacks a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

In other embodiments, the method comprises: (a) forming a reaction mixture by combining: (i) at least one entry vector as disclosed herein, wherein the methylation sites of the backbone component are unmethylated; (ii) a polynucleic acid fragment, wherein the polynucleic acid fragment comprises an internal sequence flanked by a common recognition site and corresponding cleavage site at both ends; (iii) a Type IIS restriction enzyme, wherein the Type IIS restriction enzyme recognizes the common recognition sites of the at least one entry vector and the polynucleic acid fragment; and (iv) a ligase; (b) incubating the reaction mixture for a time sufficient for Type IIS restriction enzyme-mediated cleavage of the at least one entry vector and the polynucleic acid fragment; and (c) incubating the reaction mixture for a time sufficient for the ligase to ligate the internal nucleic acid sequence of the polynucleic acid fragment with the insert component of each of the at least one entry vector, thereby generating a circular polynucleic acid; and wherein the internal sequences of the polynucleic acid fragment comprises a selectable marker and an origin of replication; and wherein the 5′ or 3′ overhangs of the insert component of each of the at least one entry vector and the internal sequence of the at least one polynucleic acid fragment uniquely complement one another so as to form a predefined sequence comprising the nucleic acid sequence of interest.

In some embodiments, the polynucleic acid fragment is a PCR product.

In some embodiments, the polynucleic acid fragment is methylated in vitro.

In some embodiments, the predefined sequence further comprises the sequence of an entry vector.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1. Schematic depicting an embodiment of a destination vector. The nucleic acid sequence of the 5′ Type IIS restriction site is CCGGTCTCNNNNNNGAGACC (SEQ ID NO: 1), and the nucleic acid sequence of the 3′ Type IIS restriction site is GGTCTCNNNNNNGAGACCGG (SEQ ID NO: 2), with N representing A, T, G, or C.

FIG. 2. Schematic depicting an embodiment of an entry vector. The nucleic acid sequence of the first Type IIS restriction site is CCGGTCTCNNNNNN (SEQ ID NO: 3), and the nucleic acid sequence of the second Type IIS restriction site is NNNNNNGAGACCGG (SEQ ID NO: 4), with N representing A, T, G, or C.

FIG. 3. Schematic depicting an embodiment of a methylation-obstructed assembly reaction. Restriction sites are as labeled in FIG. 1 and FIG. 2.

### DETAILED DESCRIPTION

Disclosed herein are compositions of destination and entry vectors, as well as the composition of kits for assembling a polynucleic acid having a predefined sequence. Also disclosed herein are methods for assembling a polynucleic acid having a predefined sequence, including methylation-obstructed assembly reactions, wherein the assembled predefined sequence may be used as an entry vector for a subsequent assembly reaction. These compositions and methods build upon previously described hierarchical cloning strategies. In particular, the disclosed compositions and methods may eliminate the need to switch restriction enzymes and/or mechanisms of selection between cloning stages. Moreover, the disclosed methods may demonstrate decreased background as the backbone of the donor vector may be degraded in the process of cloning.

### Destination Vectors and Compositions

In some aspects, the disclosure relates to destination vector polynucleic acids and compositions including destination vector polynucleic acids. In some embodiments, a destination vector is a linear vector. In other embodiments, a destination vector is a circular vector. A destination vector comprises a backbone component and an insertion site component.

As used herein, the term “backbone component” refers to the portion of a destination vector that flanks the insertion component (or the portion of an entry vector that flanks the insert component, see below in “Entry Vectors and Compositions”) and that comprises at least an origin of replication. In some embodiments, a backbone component further comprises a selectable marker gene.

In some embodiments, the selectable marker comprises a visible readout gene, such as a fluorescent or a chromogenic protein. Examples of fluorescent proteins are known to those having skill in the art and include, but are not limited to TagBFP, mTagBFP2, Azurite, EBFP2, mKalama1, Sirius, Sapphire, T-Sapphire, ECFP, Cerulean, SCFP3A, mTurquoise, mTurquoise2, monomeric Midoriishi-Cyan, TagCFP, mTFP1, EGFP, Emerald, Superfolder GFP, Monomeric Azami Green, TagGFP2, mUKG, mWasabi, Clover, mNeonGreen, EYFP, Citrine, Venus, SYFP2, TagYFP, Monomeric Kusabira-Orange, mKOK, mKO2, mOrange, mOrange2, mRaspberry, mCherry, mStrawberry, mTangerine, tdTomato, TagRFP, TagRFP-T, mApple, mRuby, mRuby2, mPlum, HcRed-Tandem, mKate2, mNeptune, NirFP, TagRFP657, IFP1.4, and iRFP. Examples of chromogenic proteins are known to those having ordinary skill in the art. See e.g., U.S. Pat. No. 9,771,402 (describing various chromogenic proteins). In some embodiments, the selectable marker comprises an auxotrophic complement gene. In some embodiments, the selectable marker comprises an antibiotic resistance gene. Examples of selectable markers are known to those having skill in the art and include, but are not limited to AmpR, NeoR, mFabI, ZeoR, NAT, HygR, SpcR (AadA), Pac, Ura3, His3, Leu2, and Trp1.

In some embodiments, a backbone component further comprises at least one Type IIS restriction site.

As used herein, the term “restriction site” refers to a polynucleic acid sequence comprising a recognition site and a corresponding cleavage site. As used herein, the term “recognition site” refers to a polynucleic acid sequence that is recognized by a Type IIS restriction enzyme and to which it specifically binds. A “corresponding cleavage site” refers to site cleaved when a Type IIS restriction enzyme binds to the recognition site. In some embodiments, a cleavage site is only two nucleotides in length, and cleavage occurs between the two nucleotides (corresponding to a blunt-end cleavage site). In some embodiments, a cleavage site is at least 3 nucleotides in length (corresponding to a 5′ or 3′ overhang site comprising at least a 1 nucleotide single-stranded overhang), at least 4 nucleotides in length (corresponding to a 5′ or 3′ overhang site comprising at least a 2 nucleotide single-stranded overhang), at least 5 nucleotides in length (corresponding to a 5′ or 3′ overhang site comprising at least a 3 nucleotide single-stranded overhang), at least 6 nucleotides in length (corresponding to a 5′ or 3′ overhang site comprising at least a 4 nucleotide single-stranded overhang), at least 7 nucleotides in length (corresponding to a 5′ or 3′ overhang site comprising at least a 5 nucleotide single-stranded overhang), at least 8 nucleotides in length (corresponding to a 5′ or 3′ overhang site comprising at least a 6 nucleotide single-stranded overhang), or at least 9 nucleotides in length (corresponding to a 5′ or 3′ overhang site comprising at least a 7 nucleotide single-stranded overhang). In some embodiments, cleavage of a cleavage site generates a single-stranded overhang. The length of the overhang may vary (e.g., at least 1, at least 2, at least 3, at least 4, at least 5, at least 6, or at least 7 nucleotides in length). In some embodiments, an overhang is a 5′ overhang. In other embodiments, an overhang is a 3′ overhang.

Type IIS restriction enzymes recognize asymmetric DNA sequences and cleave outside of their recognition sequences. Examples of known Type IIS restriction enzymes include, but are not limited to AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MboII, MlyI, MmeI, MnII, NmeAIII, PleI, SapI, and SfaNI. In some embodiments, the Type IIS restriction enzyme is a high fidelity restriction enzyme.

Some Type IIS restriction enzymes are methylation sensitive and include, but are not limited to, AlwI (dam methylation sensitive), BceAI (CpG methylation sensitive), BcgI (dam and CpG methylation sensitive), BcoDI (CpG methylation sensitive), BfuAI (CpG methylation sensitive), BsaI (dcm and CpG methylation sensitive), BsmAI (CpG methylation sensitive), BsmBI (CpG methylation sensitive), BsmFI (dcm and CpG methylation sensitive), BtgZI (CpG methylation sensitive), EarI (CpG methylation sensitive), EciI (CpG methylation sensitive), Esp3I (CpG methylation sensitive), FauI (CpG methylation sensitive), FokI (dcm and CpG methylation sensitive), HgaI (CpG methylation sensitive), HphI (dam and dcm methylation sensitive), HpyAV (CpG methylation sensitive), MboII (dam methylation sensitive), MmeI (CpG methylation sensitive), SapI (CpG methylation sensitive), PleI (CpG methylation sensitive), and SfaNI (CpG methylation sensitive).

In some embodiments, a recognition site is a common recognition site. Each “common recognition site” in a destination vector (or entry vector described below in “Entry Vectors and Compositions”): (i) consists of the same nucleic acid sequence and/or (ii) comprises a nucleic acid sequence that is recognized and bound by the same Type IIS restriction enzyme (some Type IIS restriction enzymes recognize various sequences; for example MmeI, which recognizes the sequences 5′-TCCRAC-3′, where R represents A or G). In some embodiments, at least one common recognition site of a backbone component is overlapped by a methylation site (i.e., a sequence recognized and methylated by a methyltransferase enzyme, such as a CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase). In some embodiments, a methylation site comprises a dcm, a dam and/or a CpG methylation site.

In some embodiments, the Type IIS restriction enzyme that binds to a common recognition site is selected from the group consisting of AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MboII, MlyI, MmeI, MnII, NmeAIII, PleI, SapI, and SfaNI. In some embodiments, the Type IIS restriction enzyme that binds to a common recognition site is a methylation sensitive restriction enzyme selected from the group consisting of AlwI, BceAI, BcgI, BcoDI, BfuAI, BsaI, BsmAI, BsmBI, BsmFI, BtgZI, EarI, EciI, Esp3I, FauI, FokI, HgaI, HphI, HpyAV, MboII, MmeI, PleI, SapI, and SfaNI. A methylation sensitive restriction enzyme will not cleave the cleavage site corresponding to a methylated recognition site.

In some embodiments, at least one Type IIS restriction site is located within or is flanking the selectable marker and/or the origin of replication of the backbone component.

In some embodiments, the backbone component comprises at least 2, at least 3, at least 4, at least 5, at least 6, at least 7, at least 8, at least 9, at least 10, or more than 10 Type IIS restriction sites. In some embodiments, at least 2 of the Type IIS restriction sites of a backbone component comprise a common recognition site. In some embodiments, each of the at least 2 common recognition sites of the backbone component is flanked by a methylation site.

In some embodiments, the cleavage site of at least one Type IIS restriction site in the backbone component (e.g., a cleavage site corresponding to a common recognition site) comprises a low ligation efficiency sequence content. For example, in some embodiments, the low ligation efficiency sequence comprises a blunt-end cleavage site. In other embodiments, the low ligation efficiency sequence comprises an overhang of two or fewer nucleotides. Additional examples of low efficiency ligation sequences are known in the art and include, but are not limited to, TNNA, TTTT, and AAAA. See e.g., Potapov V. et al., A single-molecule sequencing assay for the comprehensive profiling of T4 DNA ligase fidelity and bias during DNA end-joining. Nucleic Acids Res. 2018 Jul. 27; 46(13):e79; Vladimir P. et al., Optimization of Golden Gate assembly through application of ligation sequence-dependent fidelity and bias profiling. BioRxiv. 2018 May 15; doi: 10.1101/322297 the entirety of which are incorporated herein.

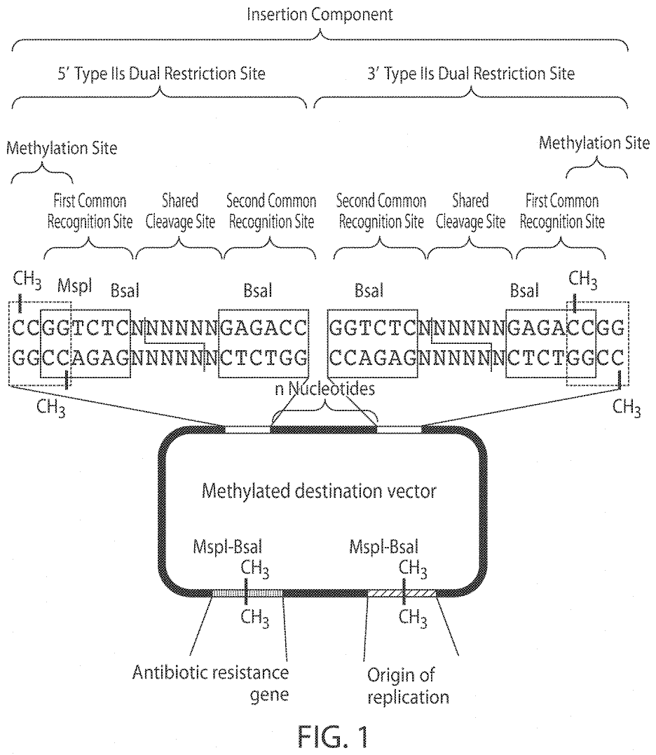

As used herein, the term “insertion site component” refers to a polynucleic acid comprising a 5′ Type IIS dual restriction site and a 3′ Type IIS dual restriction site (see e.g., FIG. 1). As used herein, the term “dual restriction site” refers to a nucleic acid sequence comprising a pair of inward facing Type IIS restriction sites (i.e., the cleavage sites corresponding to each—the first and the second—recognition site are both located between the two recognition sites). In some embodiments, the nucleic acid sequence of the 3′ Type IIS dual restriction site is the reverse complement of the 5′ Type IIS dual restriction site, with exception to the nucleic acid sequence of one or more cleavage sites. For example, in some embodiments, the nucleic acid sequence of the 5′ Type IIS dual restriction site is CCGGTCTCNNNNNNGAGACC (SEQ ID NO: 1) and the nucleic acid sequence of the 3′ Type IIS dual restriction site is GGTCTCNNNNNNGAGACCGG (SEQ ID NO: 2), with N representing A, T, G, or C.

In some embodiments, the first and/or second recognition site of a dual restriction site is a common recognition site. In some embodiments, the common recognition site of a dual restriction site is overlapped by a methylation site (i.e., a sequence recognized and methylated by a methyltransferase enzyme). In some embodiments, a methylation site comprises a dcm, a dam and/or a CpG methylation site.

In some embodiments, a dual restriction site comprises: (i) a first recognition site and corresponding cleavage site, wherein the first recognition site is overlapped by a methylation site that forms the border between the insertion site component and the backbone component and (ii) a second recognition site and corresponding cleavage site, wherein the second recognition site lacks an overlapping methylation site.

In some embodiments, the cleavage site corresponding to the first recognition site and the cleavage site corresponding to the second recognition site of a dual restriction site are separated from each other by at least 1, at least 2, at least 3, at least 4, at least 5, at least 10, at least 15, at least 20, at least 25 at least 30, at least 40, at least 50, at least 60, at least 70, at least 80, at least 90, or at least 100 nucleotides.

In some embodiments, the cleavage site corresponding to the first common recognition site and the cleavage site corresponding to the second common recognition site of the 5′ Type IIS dual restriction site or the 3′ Type IIS dual restriction site are separated from each other by at least one nucleotide. In some embodiments, the cleavage site corresponding to the first common recognition site and the cleavage site corresponding to the second common recognition site of both the 5′ Type IIS dual restriction site and the 3′ Type IIS dual restriction site are separated from each other by at least one nucleotide.

In some embodiments, the cleavage site corresponding to the first common recognition site and the cleavage site corresponding to the second common recognition site of the 5′ Type IIS dual restriction site and the cleavage site corresponding to the first common recognition site and the cleavage site corresponding to the second common recognition site of the 3′ Type IIS dual restriction site are separated from each other by an identical nucleotide sequence (i.e., the same nucleotide sequence).

In some embodiments, the cleavage site corresponding to the first common recognition site and the cleavage site corresponding to the second common recognition site of the 5′ Type IIS dual restriction site and the cleavage site corresponding to the first common recognition site and the cleavage site corresponding to the second common recognition site of the 3′ Type IIS dual restriction site are separated from each other by differing nucleotide sequences. Differing nucleotide sequences may differ in the identity of one or more nucleotide. In some embodiments, the differing nucleotide sequences are the same length. In other embodiments, the differing nucleotide sequences are different lengths.

In some embodiments, the cleavage site corresponding to the first recognition site and the cleavage site corresponding to the second recognition site of a dual restriction site comprise a shared cleavage site (i.e., a Type IIS restriction enzyme that binds to the first recognition site cleaves the same cleavage site as a Type IIS restriction enzyme that binds to the second recognition site). In some embodiments, the cleavage site corresponding to the first common recognition site and the cleavage site corresponding to the second common recognition site of the 5′ Type IIS dual restriction site or the 3′ Type IIS dual restriction site comprise a shared cleavage site. In some embodiments, the cleavage site corresponding to the first common recognition site and the cleavage site corresponding to the second common recognition site of both the 5′ Type IIS dual restriction site and the 3′ Type IIS dual restriction site comprise a shared cleavage site.

In some embodiments, the cleavage site corresponding to the first recognition site and the cleavage site corresponding to the second recognition site are identical (even though the cleavage sites are not shared).

In some embodiments, the cleavage site corresponding to the first recognition site and the cleavage site corresponding to the second recognition site differ in nucleotide sequence.

In some embodiments, the cleavage site corresponding to the first recognition site and the cleavage site corresponding to the second recognition site differ in nucleotide length. In some embodiments, the cleavage site corresponding to the first recognition site and/or the cleavage site corresponding to the second recognition site of the 5′ Type IIS dual restriction site and the first recognition site and/or the second recognition site of the 3′ Type IIS restriction site differ in length.

In some embodiments, the 5′ Type IIS dual restriction site and the 3′ Type IIS dual restriction site of an insertion site component are separated by at least 1, at least 2, at least 3, at least 4, at least 5, at least 10, at least 15, at least 20, at least 25, at least 30, at least 40, at least 50, at least 60, at least 70, at least 80, at least 90, at least 100, at least 150, at least 200, at least 300, at least 400, at least 500, at least 600, at least 700, at least 800, at least 900, at least 1000, at least 2000, or at least 5000 nucleotides. In some embodiments, the nucleic acid sequence separating the 5′ Type IIS dual restriction site and the 3′ Type IIS dual restriction site comprises a counterselectable marker (e.g., sacB, rpsL(strA), tatAR, pheS, thyA, lacY, lacZ, gata-1, ccdb, galK, or ePheSA294G). In some embodiments, the counterselectable marker is a visual readout or suicide cassette (i.e., lethal counterselector). In some embodiments, the visual readout is selected from the group consisting of a fluorescent protein or LacZ. In some embodiments, the suicide cassette comprises the nucleic acid sequence of ccdB.

In some embodiments, at least one Type IIS restriction site in the backbone component differs in nucleotide sequence from at least one Type IIS restriction site in the insertion component.

In some embodiments, at least one cleavage site corresponding to a common recognition site in the backbone component differs in nucleotide sequence from at least one cleavage site corresponding to a common recognition site in the insert component. In some embodiments, each cleavage site corresponding to a common recognition site in the backbone component differs in nucleotide sequence from each cleavage site corresponding to a common recognition site in the insert component. In some embodiments, each cleavage site corresponding to a common recognition site in the destination vector is unique.

In some embodiments, the methylation sites of each common recognition site that is overlapped by a methylation site in a destination vector are the same (i.e., each methylation site that is overlapped with a common recognition site in the destination vector can be methylated by the same methyltransferase enzyme). In some embodiments, the methylation sites of at least two common recognition site that are overlapped by a methylation sites in a destination vector are unique (i.e., at least two methylation sites that are overlapped with a common recognition site in the destination vector are methylated by different methyltransferase enzymes). Examples of methyltransferase enzymes include, but are not limited to, CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

FIG. 1 provides a schematic depicting one embodiment of a destination vector. In this embodiment, the 5′ Type IIS dual restriction site and the 3′ Type IIS dual restriction site each comprise a first common recognition site overlapped by a methylation site and a second common recognition site. In this case, the common recognition site is that of BsaI. The BsaI restriction site comprises a recognition site of 5′-GGTCTC-3′ and a cleavage site of (N1)/(N5) (i.e., 5′-NNNNNN-3′, with N representing A, T, G, or C, and generating a four base pair 5′ overhang upon cleavage). In this case the cleavage site of the first common recognition site and the second common recognition site is a shared cleavage site. One having skill in the art will recognize that the nucleic acid sequence of the shared cleavage site of the 5′ Type IIS dual restriction site and the nucleic acid sequence of the shared cleavage site of the 3′ Type IIS dual restriction site may differ from one another. One having skill in the art will also appreciate that the BsaI common recognition site (and its corresponding cleavage site) can be substituted with any other Type IIS restriction enzyme recognition site. Moreover, one having skill in the art will appreciate that the cleavage site of the first common recognition site and the cleavage site of the second common recognition site of the 5′ Type IIS dual restriction site and/or the 3′ Type IIS dual restriction site need not be a shared cleavage site. In addition, this embodiment depicts a MspI methylation site overlapping with the BsaI recognition site. One having skill in the art will appreciate that the MspI methylation site can be replaced with any other methylation site known in the art.

In some embodiments, exposure of the destination vector, when methylated, to a Type IIS restriction enzyme that recognizes the common recognition sites of the destination vector, generates at least two polynucleic acid fragments, wherein the terminal 5′ or 3′ nucleic acid overhangs of the fragment comprising the backbone component differ in nucleotide sequence.

In some embodiments, exposure of the destination vector, when unmethylated, to a Type IIS restriction enzyme that recognizes the common recognition sites of the destination vector generates at least three polynucleic acid fragments, wherein each polynucleic acid comprises terminal 5′ or 3′ nucleic acid overhangs, and wherein the terminal 5′ or 3′ nucleic acid overhangs of the fragment comprising the second common recognition sequence of both the 5′ Type IIS dual restriction site and the 3′ Type IIS dual restriction site differ in nucleotide sequence.

In some embodiments, a polynucleic acid destination vector comprising a backbone component and an insertion site component, wherein: (a) the backbone component comprises a nucleic acid sequence of a selectable marker, an origin of replication, and at least one Type IIS restriction site comprising a common recognition site and corresponding cleavage site, wherein the common recognition site is overlapped by a methylation site; and (b) the insertion site component comprises a 5′ Type IIS dual restriction site and a 3′ Type IIS dual restriction site, optionally, wherein the 5′ and 3′ Type IIS dual restriction sites are separated by at least one nucleotide; wherein each Type IIS dual restriction site comprises: (i) a first common recognition site and corresponding cleavage site, wherein the first common recognition site is overlapped by a methylation site that forms the border between the insertion site component and the backbone component and (ii) a second common recognition site and corresponding cleavage site, wherein the second recognition site lacks an overlapping methylation site, wherein the cleavage site corresponding to the first common recognition site and the cleavage site corresponding to the second common recognition site are both positioned between the first common recognition site and the second common recognition site, wherein: methylation of the destination vector at common recognition sites overlapped by a methylation site, blocks cleavage of the cleavage site correspond to that common recognition site; exposure of the destination vector, when methylated, to a Type IIS restriction enzyme that recognizes the common recognition sites of the destination vector, generates two polynucleic acid fragments, wherein the terminal 5′ or 3′ nucleic acid overhangs of the fragment comprising the backbone component differ in nucleotide sequence; and the Type IIS cleavage sites in (a) differ from the Type IIS cleavage sites in (b) in nucleotide sequence.

### Entry Vectors and Compositions

In some aspects, the disclosure relates to entry vector polynucleic acids and compositions including entry vector polynucleic acids. In some embodiments, an entry vector is a linear vector. In other embodiments, an entry vector is a circular vector. An entry vector comprises a backbone component (as described above in “Destination Vectors and Compositions”) and an insert component.

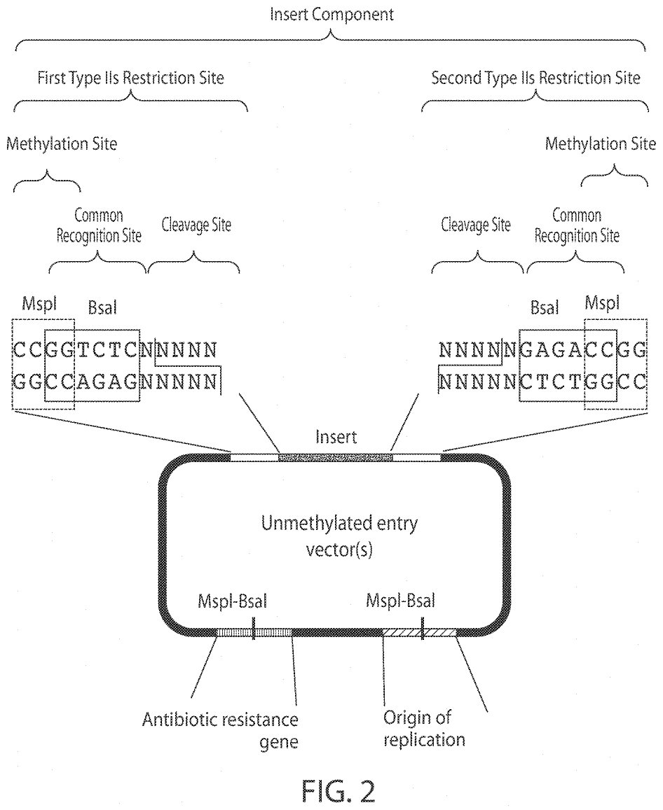

As used herein, the term “insert component” refers to a polynucleic acid comprising a first Type IIS restriction site, an insert, and a second Type IIS restriction site (see e.g., FIG. 2). As used herein, the term “insert” refers to the nucleic acid sequence flanked by the first and second Type IIS restriction site. The nucleotide length of an insert may vary. For example, an insert may be at least 20, at least 50, at least 100, at least 200, at least 500, at least 1000, at least 2000, at least 5000, at least 10,000, or at least 20,000 nucleotides in length.

In some embodiments, the first Type IIS restriction site and the second Type IIS restriction site of an insert component are inward facing (i.e., the cleavage site corresponding to the first and second recognition site is located between the two recognition sites). In some embodiments, the recognition site of the first Type IIS restriction site and the recognition site of the second Type IIS restriction site are both common recognition sites.

In some embodiments, the cleavage site of the first Type IIS restriction site of an insert component and the cleavage site of the second Type IIS restriction site of an insert component differ in nucleotide sequence.

In some embodiments, the nucleic acid sequence of the first restriction site is the reverse complement of the second restriction site, with exception to the sequence of the cleavage sites. For example, in some embodiments, the nucleic acid sequence of the first Type IIS restriction site is CCGGTCTCNNNNNN (SEQ ID NO: 3) and the nucleic acid sequence of the second Type IIS restriction site is NNNNNNGAGACCGG (SEQ ID NO: 4), with N representing A, T, G, or C.

In some embodiments, at least one Type IIS restriction site in the backbone component of an entry vector differs in nucleotide sequence from at least one Type IIS restriction site in the insertion component of an entry vector.

In some embodiments, at least one cleavage site corresponding to a common recognition site in the backbone component differs in nucleotide sequence from at least one cleavage site corresponding to a common recognition site in the insert component. In some embodiments, each cleavage site corresponding to a common recognition site in the backbone component differs in nucleotide sequence from each cleavage site corresponding to a common recognition site in the insert component. In some embodiments, each cleavage site corresponding to a common recognition site is unique.

In some embodiments, the methylation sites of each common recognition site that is overlapped by a methylation site in an entry vector are the same (i.e., each methylation site that is overlapped with a common recognition site in the destination vector can be methylated by the same methyltransferase enzyme). In some embodiments, the methylation sites of at least two common recognition site that are overlapped by methylation sites in an entry vector are unique (i.e., at least two methylation sites that are overlapped with a common recognition site in the destination vector are methylated by different methyltransferase enzymes).

In some embodiments, methylation of an entry vector at common recognition sites overlapped by a methylation site, blocks cleavage of the cleavage site correspond to that common recognition site.

In some embodiments, exposure of an entry vector, when unmethylated, to a Type IIS restriction enzyme that recognizes the common recognition sites of the entry vector generates at least two polynucleic acid fragments, wherein each polynucleic acid fragment comprises terminal 5′ or 3′ nucleic acid overhangs, and wherein one of the at least two polynucleic acid fragment comprises the insert of the entry vector. In some embodiments, the 5′ or 3′ overhangs of the polynucleic acid fragment that comprises the insert differ in nucleotide sequence.

In some embodiments, a polynucleic acid entry vector comprises a backbone component and an insert component, wherein (a) the backbone component comprises the backbone component of a polynucleic acid destination vector as disclosed herein; and (b) the insert component comprises from 5′ to 3′, a first Type IIS restriction site, an insert, and a second Type IIS restriction site; wherein the first and second Type IIS restriction sites each comprises: (i) a common recognition site overlapped by a methylation site, wherein the methylation site forms the border between the insert component and the backbone component and (ii) a corresponding cleavage site, wherein cleavage of the cleavage site of the first and second Type IIS restriction sites generates 5′ or 3′ overhangs, wherein the 5′ or 3′ overhang of the first Type IIS restriction site and the 5′ or 3′ overhang of the second Type IIS restriction sites differ in nucleotide sequence from each other.

FIG. 2 provides a schematic depicting one embodiment of an entry vector. In this embodiment, the first Type IIS restriction site and the second Type IIS dual restriction site each comprise a common recognition site overlapped by a methylation site. In this case, the common recognition site is that of BsaI. The BsaI restriction site comprises a recognition site of 5′-GGTCTC-3′ and a cleavage site of (N1)/(N5) (i.e., 5′-NNNNNN-3′, with N representing A, T, G, or C, and generating a four base pair 5′ overhang upon cleavage). One having skill in the art will appreciate that the BsaI common recognition site (and its corresponding cleavage site) can be substituted with that of any other Type IIS restriction enzyme recognition site. Moreover, one having skill in the art will appreciate that the cleavage site of the first Type IIS restriction site and the cleavage site of the second Type IIS restriction site need not be identical. In addition, this embodiment depicts a MspI methylation site overlapping with the BsaI recognition site. One having skill in the art will appreciate that the MspI methylation site can be replaced with any number of methylation sites known in the art.

### Compositions of Kits for Assembling a Polynucleic Acid Having a Predefined Sequence

In some aspects, the disclosure relates to compositions of kits for the assembly of polynucleic acids having a predefined sequence. In some embodiments, the kit comprises a set of destination vectors, as described above in “Destination Vectors and Compositions,” wherein: (i) the cleavage site corresponding to the first common recognition site of the 5′ Type IIS dual restriction site and the cleavage site corresponding to the first recognition site of the 3′ Type IIS dual restriction site of each destination vector in the set of destination vectors differ in nucleotide sequence; (ii) the cleavage site of the 5′ Type IIS dual restriction site of at least one destination vector in the set of destination vectors is identical to the 3′ Type IIS dual restriction site in at least one other destination vector in the set of destination vectors; and (iii) the cleavage site of the 3′ Type IIS dual restriction site of at least one destination vector in the set of destination vectors is identical to the 5′ Type IIS dual restriction site in at least one other destination vector in the set of destination vectors.

In some embodiments, each destination vector in the set of destination vectors comprises a backbone component and an insertion site component, wherein: (a) the backbone component comprises a nucleic acid sequence of a selectable marker, an origin of replication, and at least one Type IIS restriction site comprising a common recognition site and corresponding cleavage site, wherein the common recognition site is overlapped by a methylation site; and (b) the insertion site component comprises a 5′ Type IIS dual restriction site and a 3′ Type IIS dual restriction site, optionally, wherein the 5′ and 3′ Type IIS dual restriction sites are separated by at least one nucleotide; wherein each Type IIS dual restriction site comprises: (i) a first common recognition site and corresponding cleavage site, wherein the first common recognition site is overlapped by a methylation site that forms the border between the insertion site component and the backbone component and (ii) a second common recognition site and corresponding cleavage site, wherein the second recognition site lacks an overlapping methylation site, wherein the cleavage site corresponding to the first common recognition site and the cleavage site corresponding to the second common recognition site are both positioned between the first common recognition site and the second common recognition site, wherein: methylation of the destination vector at common recognition sites overlapped by a methylation site, blocks cleavage of the cleavage site correspond to that common recognition site; exposure of the destination vector, when methylated, to a Type IIS restriction enzyme that recognizes the common recognition sites of the destination vector, generates two polynucleic acid fragments, wherein the terminal 5′ or 3′ nucleic acid overhangs of the fragment comprising the backbone component differ in nucleotide sequence; and the Type IIS cleavage sites in (a) differ from the Type IIS cleavage sites in (b) in nucleotide sequence.

In some embodiments, the set of destination vectors comprises at least 3, at least 4, at least 5, at least 6, at least 7, at least 8, at least 9, at least 10, at least 11, at least 12, at least 13, at least 14, at least 15, at least 16, at least 17, at least 18, at least 20, at least 25, at least 30, at least 40, at least 50, or more than 50 different destination vectors. In some embodiments each of the destination vectors in the set of destination vectors comprise the same backbone component. In some embodiments, at least one destination vector in the set of destination vectors comprises a unique backbone component.

In some embodiments, the kit further comprises at least one reaction buffer (e.g., a digestion buffer, a ligase buffer, a methyltransferase buffer, and/or a universal buffer), at least one Type IIS restriction enzyme (e.g., AlwI, BbsI, BceAI, BcgI, BcoDI, BfuAI, BsaI, BsmAI, BsmBI, BsmFI, BtgZI, EarI, EciI, Esp3I, FauI, FokI, HgaI, HphI, HpyAV, MboII, MmeI, PleI, SapI, SfaNI, and/or functional variants thereof), at least one methyltransferase (e.g., CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, TaqI methyltransferase, and/or functional variants thereof), at least one ligase (e.g., T3 DNA ligase, T4 DNA ligase, T7 DNA ligase, E. coli DNA ligase, taq DNA ligase, and/or functional variants thereof), and/or at least one preparation of competent cells.

In some embodiments, the kit comprises at least two preparations of competent cells, wherein at least one preparation of competent cells comprises cells that express a methyltransferase capable of methylating the destination vector at methylation sites overlapping common recognition sites, and wherein at least one preparation of competent cells comprises cells that are incapable of methylating the destination vector at methylation sites overlapping common recognition sites. In some embodiments the competent cells are prokaryotic cells. In some embodiments, the competent cells are eukaryotic cells.

### Methods of Assembling a Polynucleic Acid Having a Predefined Sequence

In some aspects, the disclosure relates to methods of assembling a polynucleic acid having a predefined sequence. In some embodiments, a method of assembling a polynucleic acid having a predefined sequence comprises forming a first reaction mixture, wherein the first reaction mixture comprises at least two polynucleic acids and a Type IIS restriction enzyme, wherein the formation of the first reaction mixture results in the generation of at least two polynucleic acid cleavage products comprising 5′ or 3′ overhangs, and wherein the at least two polynucleic acid cleavage products together comprise the polynucleic acid having the predefined sequence. In some embodiments, the method further comprises forming a second reaction mixture, wherein the second reaction mixture comprises the at least two polynucleic acid cleavage products and a ligase, wherein the 5′ or 3′ overhangs of the at least two polynucleic acid cleavage products uniquely complement one another so as to form a predefined sequence through ligation.

In some embodiments, the first reaction mixture and the second reaction mixture are formed sequentially (i.e., the reaction mixture comprising the Type IIS restriction enzyme is formed first, followed by the reaction mixture comprising the ligase). In some embodiments, the at least two polynucleic acid cleavage products are purified prior to forming the second reaction mixture. In some embodiments, the first reaction mixture and the second reaction mixture are the same (i.e., cleavage and ligation of destination and entry vectors occurs in a single reaction volume).

In some embodiments, the at least two polynucleic acids of the first reaction mixture comprise a destination vector, as described above in “Destination Vectors and Compositions,” and at least one entry vector, as described above in “Entry Vectors and Compositions.” For example, in some embodiments, a method comprises: (a) forming a reaction mixture by combining: (i) a destination vector, wherein the methylation sites of both the backbone component and the insertion site component of the destination vector are methylated; (ii) at least one entry vector, wherein the methylation sites of the backbone component and the insert component of each of the at least one entry vectors are unmethylated; (iii) a Type IIS restriction enzyme, wherein the Type IIS restriction enzyme recognizes the common recognition sites of the destination vector and entry vector; and (iv) a ligase; (b) incubating the reaction mixture for a time sufficient for Type IIS restriction enzyme-mediated cleavage of the destination vector and the at least one entry vectors; and (c) incubating the reaction mixture for a time sufficient for the ligase to ligate the insert of each of the at least one entry vector into the backbone component of the destination vector, thereby generating a circular polynucleic acid; and wherein the 5′ or 3′ overhangs of the backbone component of the destination vector and the insert component of each of the at least one entry vector uniquely complement one another so as to form a predefined sequence comprising the backbone component of the destination vector of step (a)(i) and the insert component of each of the at least one entry vectors of step (a)(ii).

In some embodiments, the first reaction mixture comprises at least 2, at least 3, at least 4, at least 5, at least 6, at least 7, at least 8, at least 9, at least 10, or more than 10 entry vectors.

In some embodiments, the destination vector is methylated in vitro (e.g., by forming a reaction mixture comprising the destination vector and a methyltransferase enzyme and incubating the reaction mixture for a time sufficient for the methyltransferase enzyme to methylate the destination vector). In other embodiments, the destination vector is methylated in vivo. For example, in some embodiments, the destination vector is methylated in a bacterial strain that expresses a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

In some embodiments, the method further comprises isolating the ligated destination vector containing the insert from the other components of the reaction mixture. In some embodiments, the ligated destination vector is isolated by transforming bacteria with the second reaction mixture and screening the bacteria for the presence of the correctly ligated assembly.

In some embodiments, the method further comprises demethylating the isolated ligated destination vector to generate a second entry vector. In some embodiments, the isolated ligated destination vector is demethylated passively. For example, the destination vector may be passively demethylated via amplification in vitro (e.g., PCR). Alternatively, the destination vector may be passively demethylated in vivo through replication in a bacterial strain that lacks a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

In some embodiments, the backbone of the destination vector and the backbone of at least one entry vector are identical. In some embodiments, the backbone of the destination vector and the backbone of each entry vector are identical.

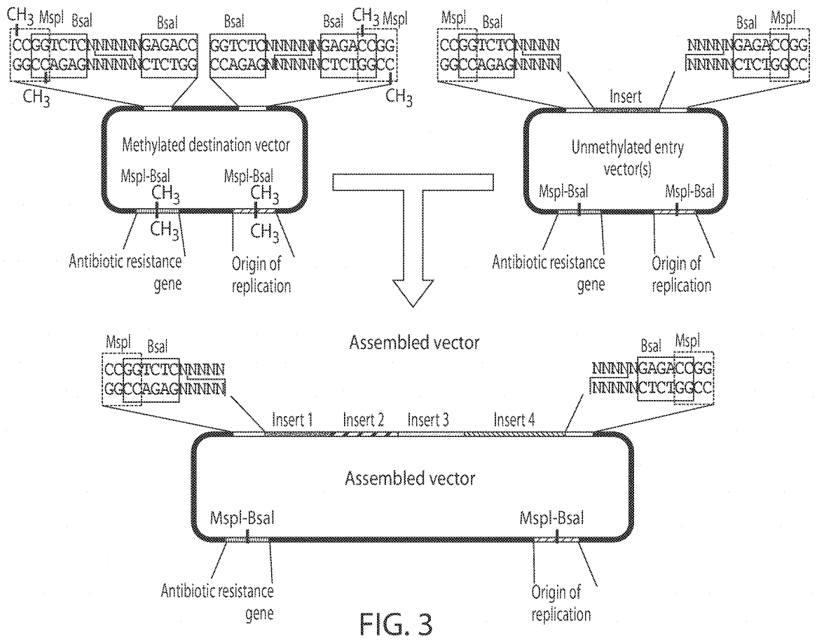

FIG. 3 provides a schematic depicting one embodiment, wherein the at least two polynucleic acids of the first reaction mixture comprise a destination vector, as described above, and at least one entry vector, as described above. One having skill in the art would appreciate that the method depicted could be modified through the use of a different destination vector and/or entry vector.

In other embodiments, the at least two polynucleic acids of the first reaction mixture comprise a destination vector, as described above in “Destination Vectors and Compositions,” and at least one polynucleic acid fragment (e.g., a PCR product or other synthetic fragment). For example, in some embodiments a method comprises: (a) forming a reaction mixture by combining: (i) a destination vector, wherein the methylation sites of the backbone component are methylated; (ii) at least one polynucleic acid fragment, wherein each polynucleic acid fragment comprises an internal sequence flanked by a common recognition site and corresponding cleavage site at both ends; (iii) a Type IIS restriction enzyme, wherein the Type IIS restriction enzyme recognizes the common recognition sites of the destination vector and the at least one polynucleic acid fragment; and (iv) a ligase; (b) incubating the reaction mixture for a time sufficient for Type IIS restriction enzyme-mediated cleavage of the destination vector and the at least one polynucleic acid fragment; and (c) incubating the reaction mixture for a time sufficient for the ligase to ligate the internal nucleic acid sequence of each of the at least one polynucleic acid fragments into the backbone component of the destination vector, thereby generating a circular polynucleic acid; and wherein the internal sequences of the at least one polynucleic acid fragment comprises a nucleic acid sequence of interest; and wherein the 5′ or 3′ overhangs of the backbone component of the destination vector and each internal sequence of the at least one polynucleic acid fragment uniquely complement one another so as to form a predefined sequence comprising the backbone component of the destination vector of step (a)(i) and the nucleic acid sequence of interest.

In some embodiments, the destination vector is methylated in vitro (e.g., by forming a reaction mixture comprising the destination vector and a methyltransferase enzyme and incubating the reaction mixture for a time sufficient for the methyltransferase enzyme to methylate the destination vector). In other embodiments, the destination vector is methylated in vivo. For example, in some embodiments, the destination vector is methylated in a bacterial strain that expresses a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

In some embodiments, the method further comprises isolating the ligated destination vector containing the insert from the other components of the second reaction mixture. In some embodiments, the ligated destination vector is isolated by transforming cells (e.g., bacteria) with the second reaction mixture and screening the cells or the progeny of the cells for the presence of the correctly ligated assembly.

In some embodiments, the method further comprises demethylating the isolated ligated destination vector to generate a second entry vector. In some embodiments, the isolated ligated destination vector is demethylated passively in vivo through replication in a bacterial strain that lacks a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

In other embodiments, the at least two polynucleic acids of the first reaction mixture comprise an entry vector, as described above in “Entry Vectors and Compositions,” and a polynucleic acid fragment (e.g., a PCR product or other synthetic fragment). For example, in some embodiments a method comprises: (a) forming a reaction mixture by combining: (i) at least one entry vector as disclosed herein, wherein the methylation sites of the backbone component are unmethylated; (ii) a polynucleic acid fragment, wherein the polynucleic acid fragment comprises an internal sequence flanked by a common recognition site and corresponding cleavage site at both ends; (iii) a Type IIS restriction enzyme, wherein the Type IIS restriction enzyme recognizes the common recognition sites of the at least one entry vector and the polynucleic acid fragment; and (iv) a ligase; (b) incubating the reaction mixture for a time sufficient for Type IIS restriction enzyme-mediated cleavage of the at least one entry vector and the polynucleic acid fragment; and (c) incubating the reaction mixture for a time sufficient for the ligase to ligate the internal nucleic acid sequence of the polynucleic acid fragment with the insert component of each of the at least one entry vector, thereby generating a circular polynucleic acid; and wherein the internal sequences of the polynucleic acid fragment comprises a selectable marker and an origin of replication; and wherein the 5′ or 3′ overhangs of the insert component of each of the at least one entry vector and the internal sequence of the at least one polynucleic acid fragment uniquely complement one another so as to form a predefined sequence comprising the nucleic acid sequence of interest.

In some embodiments, the first reaction mixture comprises at least 2, at least 3, at least 4, at least 5, at least 6, at least 7, at least 8, at least 9, at least 10, or more than 10 entry vectors.

In some embodiments, the backbone of each entry vector is identical.

In some embodiments, the polynucleic acid fragment comprises the sequence of a backbone component of a destination vector or entry vector, as described above. In some embodiments, the polynucleic acid fragment is methylated in vitro.

In some embodiments, the predefined sequence further comprises the sequence of an entry vector.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A polynucleic acid destination vector comprising a backbone component and an insertion site component, wherein:
(a) the backbone component comprises a nucleic acid sequence of a selectable marker, an origin of replication, and at least one Type IIS restriction site comprising a common recognition site and corresponding cleavage site, wherein the common recognition site is overlapped by a methylation site; and
(b) the insertion site component comprises a 5′ Type IIS dual restriction site and a 3′ Type IIS dual restriction site, optionally, wherein the 5′ and 3′ Type IIS dual restriction sites are separated by at least one nucleotide; wherein each Type IIS dual restriction site comprises: (i) a first common recognition site and corresponding cleavage site, wherein the first common recognition site is overlapped by a methylation site that forms the border between the insertion site component and the backbone component and (ii) a second common recognition site and corresponding cleavage site, wherein the second recognition site lacks an overlapping methylation site, wherein the cleavage site corresponding to the first common recognition site and the cleavage site corresponding to the second common recognition site are both positioned between the first common recognition site and the second common recognition site, wherein:
methylation of the destination vector at common recognition sites overlapped by a methylation site, blocks cleavage of the cleavage site corresponding to that common recognition site;
exposure of the destination vector, when methylated, to a Type IIS restriction enzyme that recognizes the common recognition sites of the destination vector, generates two polynucleic acid fragments, wherein the terminal 5′ or 3′ nucleic acid overhangs of the fragment comprising the backbone component differ in nucleotide sequence; and
the Type IIS cleavage sites in (a) differ from the Type IIS cleavage sites in (b) in nucleotide sequence.

2. The destination vector of claim 1, wherein the cleavage site corresponding to the first common recognition site and the cleavage site corresponding to the second common recognition site of the 5′ Type IIS dual restriction site or the 3′ Type IIS dual restriction site are separated from each other by at least one nucleotide.

3. The destination vector of claim 1, wherein the cleavage site corresponding to the first common recognition site and the cleavage site corresponding to the second common recognition site of both the 5′ Type IIS dual restriction site and the 3′ Type IIS dual restriction site are separated from each other by at least one nucleotide.

4. The destination vector of claim 3, wherein the cleavage site corresponding to the first common recognition site and the cleavage site corresponding to the second common recognition site of the 5′ Type IIS dual restriction site and the cleavage site corresponding to the first common recognition site and the cleavage site corresponding to the second common recognition site of the 3′ Type IIS dual restriction site are separated from each other by differing nucleotide sequences.

5. The destination vector of claim 3, wherein the differing nucleotide sequences comprise differing nucleic acid lengths.

6. The destination vector of claim 3, wherein the cleavage site corresponding to the first common recognition site and the cleavage site corresponding to the second common recognition site of the 5′ Type IIS dual restriction site and the cleavage site corresponding to the first common recognition site and the cleavage site corresponding to the second common recognition site of the 3′ Type IIS dual restriction site are separated from each other by an identical nucleotide sequence.

7. The destination vector of claim 1, wherein the cleavage site corresponding to the first common recognition site and the cleavage site corresponding to the second common recognition site of the 5′ Type IIS dual restriction site and/or the 3′ Type IIS dual restriction site is a shared cleavage site.

8. The destination vector of any one of claims 1-7, wherein the 5′ Type IIS dual restriction site and a 3′ Type IIS dual restriction site are separated by a nucleic acid sequence encoding a visual readout or suicide cassette.

9. The destination vector of claim 8, wherein the visual readout is selected from the group consisting of a fluorescent protein, a chromogenic protein, LacZ, or LacZa.

10. The destination vector of claim 8, wherein the suicide cassette comprises the nucleic acid sequence of ccdB.

11. The destination vector of any one of claims 1-10, wherein the Type IIS restriction enzyme that binds to the common recognition site is selected from the group consisting of BsaI, BsmBI, BtgZI, Esp3I, FokI, HphI, BcgI, AlwI, MboII, MmeI, BsmFI, BceAI, BcoDI, BfuAI, BsmAI, EarI, EciI, FauI, HgaI, HpyAV, PleI, BbsI, SapI, and SfaNI.

12. The destination vector of any one of claims 1-11, wherein at least one Type IIS restriction site in the backbone component of (a) is located within or flanking the selectable marker or the origin of replication.

13. The destination vector of any one of claims 1-12, wherein the cleavage site of at least one Type IIS restriction site in the backbone component of (a) comprises a low ligation efficiency sequence content.

14. The destination vector of any one of claims 1-13, wherein the selectable marker comprises an antibiotic resistance gene.

15. The destination vector of any one of claims 1-14, wherein the methylation sites of (a) and (b) are methylated by the same methyltransferase.

16. The destination vector of claim 15, wherein the methyltransferase is selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

17. The destination vector of any one of claims 1-16, wherein exposure of the destination vector, when unmethylated, to a Type IIS restriction enzyme that recognizes the common recognition sites of the destination vector generates at least three polynucleic acid fragments, wherein each polynucleic acid comprises terminal 5′ or 3′ nucleic acid overhangs, and wherein the terminal 5′ or 3′ nucleic acid overhangs of the fragment comprising the second common recognition sequence of both the 5′ Type IIS dual restriction site and the 3′ Type IIS dual restriction site differ in nucleotide sequence.

18. A polynucleic acid entry vector comprising a backbone component and an insert component, wherein
(a) the backbone component comprises the backbone component of a polynucleic acid destination vector of any one of claims 1-17; and
(b) the insert component comprises from 5′ to 3′, a first Type IIS restriction site, an insert, and a second Type IIS restriction site; wherein the first and second Type IIS restriction sites each comprises: (i) a common recognition site overlapped by a methylation site, wherein the methylation site forms the border between the insert component and the backbone component and (ii) a corresponding cleavage site, wherein cleavage of the cleavage site of the first and second Type IIS restriction sites generates 5′ or 3′ overhangs, wherein the 5′ or 3′ overhang of the first Type IIS restriction site and the 5′ or 3′ overhang of the second Type IIS restriction sites differ in nucleotide sequence from each other.

19. The entry vector of claim 18, wherein the insert is a nucleic acid sequence that is to be combined in an assembly reaction.

20. A method for assembling polynucleic acids into a predefined sequence, said method comprising:
(a) forming a reaction mixture by combining: (i) a destination vector of any one of claims 1-17, wherein the methylation sites of both the backbone component and the insertion site component of the destination vector are methylated; (ii) at least one entry vector of any one of claims 18-19, wherein the methylation sites of the backbone component and the insert component of each of the at least one entry vectors are unmethylated; (iii) a Type IIS restriction enzyme, wherein the Type IIS restriction enzyme recognizes the common recognition sites of the destination vector and entry vector; and (iv) a ligase;
(b) incubating the reaction mixture for a time sufficient for Type IIS restriction enzyme-mediated cleavage of the destination vector and the at least one entry vectors; and
(c) incubating the reaction mixture for a time sufficient for the ligase to ligate the insert of each of the at least one entry vector into the backbone component of the destination vector, thereby generating a circular polynucleic acid; and
wherein the 5′ or 3′ overhangs of the backbone component of the destination vector and the insert component of each of the at least one entry vector uniquely complement one another so as to form a predefined sequence comprising the backbone component of the destination vector of step (a)(i) and the insert component of each of the at least one entry vectors of step (a)(ii).

21. The method of claim 20, wherein the destination vector is methylated in vitro.

22. The method of claim 20, wherein the destination vector is methylated in vivo.

23. The method of claim 22, wherein the destination vector is methylated in a bacterial strain that expresses a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

24. The method of any one of claims 20-23, further comprising isolating the ligated destination vector containing the insert from the other components of the reaction mixture.

25. The method of claim 24, wherein the ligated destination vector is isolated by transforming bacteria with the reaction mixture and screening the bacteria for the presence of the correctly ligated assembly.

26. The method of any one of claims 20-25, further comprising demethylating the isolated ligated destination vector to generate a second entry vector.

27. The method of claim 26, wherein the isolated ligated destination vector is demethylated passively through replication in a bacterial strain that lacks a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

28. A method of cloning a nucleic acid sequence of interest, said method comprising:
(a) forming a reaction mixture by combining: (i) a destination vector of any one of claims 1-17, wherein the methylation sites of the backbone component are methylated; (ii) at least one polynucleic acid fragment, wherein each polynucleic acid fragment comprises an internal sequence flanked by a common recognition site and corresponding cleavage site at both ends; (iii) a Type IIS restriction enzyme, wherein the Type IIS restriction enzyme recognizes the common recognition sites of the destination vector and the at least one polynucleic acid fragment; and (iv) a ligase;
(b) incubating the reaction mixture for a time sufficient for Type IIS restriction enzyme-mediated cleavage of the destination vector and the at least one polynucleic acid fragment; and
(c) incubating the reaction mixture for a time sufficient for the ligase to ligate the internal nucleic acid sequence of each of the at least one polynucleic acid fragments into the backbone component of the destination vector, thereby generating a circular polynucleic acid; and
wherein the internal sequences of the at least one polynucleic acid fragment comprises a nucleic acid sequence of interest; and
wherein the 5′ or 3′ overhangs of the backbone component of the destination vector and each internal sequence of the at least one polynucleic acid fragment uniquely complement one another so as to form a predefined sequence comprising the backbone component of the destination vector of step (a)(i) and the nucleic acid sequence of interest.

29. The method of claim 28, wherein the destination vector is methylated in vitro.

30. The method of claim 28, wherein the destination vector is methylated in vivo.

31. The method of claim 30, wherein the destination vector is methylated in a bacterial strain that expresses a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

32. The method of any one of claims 28-31, further comprising isolating the ligated destination vector containing the predefined sequence from the other components of the reaction mixture.

33. The method of claim 32, wherein the ligated destination vector is isolated by transforming bacteria with the reaction mixture and screening the bacteria for the presence of the correctly ligated assembly.

34. The method of any one of claims 28-33, further comprising demethylating the isolated ligated destination vector to generate a second entry vector.

35. The method of claim 34, wherein the isolated ligated destination vector is demethylated passively in vivo through replication in a bacterial strain that lacks a methyltransferase selected from the group consisting of CpG methyltransferase (optionally M.SssI), dam methyltransferase, dcm methyltransferase, GpC methyltransferase (optionally M.CviPI), AluI methyltransferase, BamHI methyltransferase, EcoRI methyltransferase, HaeIII methyltransferase, HhaI methyltransferase, HpaII methyltransferase, MspI methyltransferase, and TaqI methyltransferase.

36. A method of cloning a nucleic acid sequence of interest, said method comprising:
(a) forming a reaction mixture by combining: (i) at least one entry vector of any one of claims 1-2, wherein the methylation sites of the backbone component are unmethylated; (ii) a polynucleic acid fragment, wherein the polynucleic acid fragment comprises an internal sequence flanked by a common recognition site and corresponding cleavage site at both ends; (iii) a Type IIS restriction enzyme, wherein the Type IIS restriction enzyme recognizes the common recognition sites of the at least one entry vector and the polynucleic acid fragment; and (iv) a ligase;
(b) incubating the reaction mixture for a time sufficient for Type IIS restriction enzyme-mediated cleavage of the at least one entry vector and the polynucleic acid fragment; and
(c) incubating the reaction mixture for a time sufficient for the ligase to ligate the internal nucleic acid sequence of the polynucleic acid fragment with the insert component of each of the at least one entry vector, thereby generating a circular polynucleic acid; and
wherein the internal sequences of the polynucleic acid fragment comprises a selectable marker and an origin of replication; and
wherein the 5′ or 3′ overhangs of the insert component of each of the at least one entry vector and the internal sequence of the at least one polynucleic acid fragment uniquely complement one another so as to form a predefined sequence comprising the nucleic acid sequence of interest.

37. The method of claim 36, wherein the polynucleic acid fragment is a PCR product.

38. The method of claim 36 or 37, wherein the polynucleic acid fragment is methylated in vitro.

39. The method of any one of claims 36-38, wherein the predefined sequence further comprises the sequence of an entry vector.


SEQUENCE LISTING (4 sequences)

SEQ ID NO: 1 - DNA, 20 bp, Artificial Sequence
  Synthetic polynucleotide
  misc_feature (9)..(14): n is a, c, g, or t
ccggtctcnnnnnngagacc

SEQ ID NO: 2 - DNA, 20 bp, Artificial Sequence
  Synthetic polynucleotide
  misc_feature (7)..(12): n is a, c, g, or t
ggtctcnnnnnngagaccgg

SEQ ID NO: 3 - DNA, 14 bp, Artificial Sequence
  Synthetic polynucleotide
  misc_feature (9)..(14): n is a, c, g, or t
ccggtctcnnnnnn

SEQ ID NO: 4 - DNA, 14 bp, Artificial Sequence
  Synthetic polynucleotide
  misc_feature (1)..(6): n is a, c, g, or t
nnnnnngagaccgg